(19) United States
(12) Patent Application Publication
(10) Pub. No.: US 2017/0112677 A1
(43) Pub. Date: Apr. 27, 2017

(54) COMPOSTABLE ABSORBENT ARTICLE

(71) Applicant: LB LYOPHARM S.R.L., Bolzano (IT)

(72) Inventor: Christian REBIÈRE, Bolzano (IT)

(21) Appl. No.: 15/315,585

(22) PCT Filed: Jun. 3, 2015
(86) PCT No.: PCT/IB2015/054189

§ 371 Date: Dec. 1, 2016

(30) Foreign Application Priority Data
Jun. 6, 2014 (IT) RM2014A000294

(51) Int. Cl. A61F 13/15; A61L 15/18; A61F 13/84; A61L 15/28; A61F 13/511; A61F 13/514; A61F 13/53; A61L 15/62

(52) U.S. Cl. CPC A61F 13/15252; A61F 13/53; A61L 15/18; A61L 15/62; A61L 15/28; A61F 13/51121; A61F 13/51401; A61F 13/8405; A61F 2013/1526; A61F 2013/8414; A61F 2013/8408; A61F 2013/51004

(57) ABSTRACT

The present invention relates to a disposable absorbent article that can be fully compostable, wherein such article contains, as sole absorbent material, one or more silicon-based products selected from: hydrated aluminosilicates and fumed silica, optionally in combination with other silica-based compounds and/or natural silica, processes for the preparation of such article and for the preparation of said absorbent material.

The present invention relates to a disposable absorbent article that can be fully compostable, wherein such article contains, as sole absorbent material, one or more silicon-based products selected from: hydrated aluminosilicates and fumed silica, optionally in combination with other silica-based compounds and/or natural silica, and to processes for the preparation of such article and for the preparation of said absorbent material.

### STATE OF THE PRIOR ART

One of the main problems manifested by disposable absorbent articles (diapers, adult diapers, feminine hygiene absorbent articles, towels, underpads for beds, cots, cribs, seats, etc.) lies in the fact that, in most cases, they are composed of numerous parts of non-natural plastic material, comprising the absorbing layer, normally containing polymers having a high absorption ability, which are not recyclable, biodegradable and/or compostable.

Currently, the sole way to dispose of these products is to destine them for an incineration plant or incinerator, which obviously entails pollution, at least in terms of fumes, or to particular and specific treatment plants in which selection processes are carried out which can be onerous or, at worst, are destined for a dump.

It is known that by now plastic is present also in suspension in sea or lake waters, and the problem of its disposal has become an issue at global level.

Given the use, it also virtually global, of disposable absorbent articles, practically in all age brackets of the human race, from infancy to old age, it is important to design and develop completely biodegradable and/or compostable absorbent articles to be intended for the end production of biogas and compost for agricultural use or for other uses, having features of absorption and feeling of dryness for the user comparable to, or even better than the products currently in use.

### SUMMARY OF THE INVENTION

The present invention provides disposable absorbent articles that can be completely compostable, in which such articles contain, as sole absorbent material, one or more silica-based inorganic products selected from: hydrated aluminosilicates and fumed silica, optionally in combination with other silica-based compounds and/or natural silica, processes for the preparation of such articles and for the preparation of said absorbent material. The Authors of the present invention have in fact found that the absorbent material as described herein exhibits absorption features making it comparable to, if not better than the absorbent polymers currently on the market, and fluid entrapment features making it even better than such absorbent polymers.

Therefore, the present invention relates to a disposable absorbent article comprising a permeable upper layer, a non-permeable lower layer and an absorbent core placed between the upper layer and the lower layer wherein said core contains, as sole absorbent material, one or more silicon-based products selected from: hydrated aluminosilicates and fumed silica, optionally in combination with other silica-based compounds and/or natural silica.

The present invention also relates to a process for the preparation of a disposable absorbent article wherein an absorbent core comprised of one or more silicon-based products selected from: hydrated aluminosilicates and fumed silica, optionally in combination with other silica-based compounds and/or natural silica, is placed between a permeable upper layer and a non-permeable lower layer and wherein said absorbent core is prepared according to the following steps:

a. a volume of absorbent material consisting of said hydrated aluminosilicates and fumed silica, optionally in combination with other silica-based compounds and/or natural silica, is mixed and rehydrated with 2-4 volumes of water and/or in combination with alginate;

b. the mixture thus obtained is dried under vacuum at a pressure of between −0.8 and −1 bar and at a temperature comprised between 150° C. and 220° C. for a period of from 1.5 to 3 hours and a disposable absorbent article obtainable by the above-described process.

Finally, the present invention also relates to a process for the preparation of an absorbent mixture comprising the following steps:

a. a volume of absorbent material consisting of hydrated aluminosilicates and fumed silica, optionally in combination with other silica-based compounds and/or natural silica and/or in combination with alginate, is mixed and rehydrated with 2-4 volumes of water;

b. the mixture thus obtained is dried under vacuum at a pressure of between −0.8 and −1 bar and at a temperature comprised between 150° C. and 220° C. for a period of from 1.5 to 3 hours and an absorbent mixture obtainable by the above-described process.

### DETAILED DESCRIPTION OF THE INVENTION

According to an aspect, the present invention relates to a disposable absorbent article comprising a permeable upper layer, a non-permeable lower layer and an absorbent core placed between the upper layer and the lower layer wherein said core contains, as sole absorbent material, one or more silicon-based products selected from: hydrated aluminosilicates and fumed silica, optionally in combination with other silica-based compounds and/or natural silica and/or in combination with alginate.

According to a particular embodiment of the above-described invention, said hydrated aluminosilicates and fumed silica, optionally in combination with alginate, represent at least 80% by weight, 90% by weight, 95% by weight, 98% by weight of said absorbent material.

In a particular embodiment of the invention the absorbent material contained in the disposable absorbent article can consist in a mixture of one or more hydrated aluminosilicates and one or more fumed silica without further materials added.

As indicated above, therefore, according to the invention one or more hydrated aluminosilicates and one or more fumed silica, in combination with other silica-based compounds and/or other minerals, can constitute the absorbent material of the disposable article described herein.

In particular, to the ends of the present invention, by the term “other silica-based compounds” there are meant silica (SiO₂)-based compounds that can be selected, e.g., in the group comprising

silicon dioxide SiO₂ bonded to other atoms such as Aluminum, Iron, Magnesium, Calcium, Sodium, Potassium.

As is known to a person skilled in the art, hydrated aluminosilicates are crystals with a tetrahedral three-dimensional scaffold. Depending on the type of structure, they contain regular channels or interconnected pores. The pores contain water and cations required to balance the excess of charge of the scaffold. The cations are mobile and can be exchanged with other cations.

Suitable for the purposes of carrying out of the present invention are aluminosilicates belonging to the family of ANALCIME, CHABASITE,

GISMONDINE, HEULANDITE, NATROLITE, STILBITE, and zeolites, which are hydrated aluminosilicates of alkali and alkali-earth metal having exclusive chemico-physical properties due to the unusual crystalline structure of zeolites, characterized by the presence of cavities and channels housing ions and water molecules having extreme mobility.

Therefore, according to the invention, the hydrated aluminosilicates can be selected, in any of the embodiments described herein, from natural or synthetic zeolites.

Silica suitable for the carrying out of the invention are, e.g., fumed silica having a specific surface area from 100 to 500 m²/g, in particular from 200 to 380 m²/g (extremes included). A non-limiting example of suitable silica are the AEROSIL® silica, which are fumed silica available on the market, normally used also as pharmaceutical excipients, and therefore considered suitable for human alimentary use. Therefore, for the carrying out of the invention silica in the above-indicated specific surface range, like, e.g., AEROSIL ® 150, 200, 250, 300, 380 or alike products could be used.

According to the invention, in any embodiment described herein there could be used, e.g., fumed silica having a specific surface area from 100 to 500 m²/g or from 200 to 380 m²/g.

According to what described above, in a particular embodiment, the hydrated aluminosilicates are selected from natural or synthetic zeolites and the fumed silica are selected from fumed silica having a specific surface area from 100 to 500 m²/g or from 200 to 380 m²/g.

In case the absorbent material be comprised of zeolites and fumed silica, such components could be present in said material in different mutual ratios.

For instance, according to different embodiments, absorbent materials are possible wherein said zeolites represent from 50% to 70% by weight of said absorbent material and said fumed silica represent from 30 to 50% by weight of said absorbent material or wherein said zeolites represent from 55% to 65% by weight of said absorbent material and said fumed silica represent from 35 to 45% by weight of said absorbent material or wherein said zeolites represent about 60% by weight of said absorbent material and said fumed silica represent about 40% by weight of said absorbent material.

In a further embodiment, the article as described above will also comprise an alginate in a variable amount of from 30% and 50% by weight of said absorbent material.

Said alginate may be in the presence or in the absence of said other silica-based compounds and/or natural silica as described above.

According to these embodiments, therefore, when alginate is present, said hydrated aluminosilicates represent from 15% to 35% by weight of said absorbent material and said alginate represents from 30% to 50% by weight of said absorbent material and said fumed silica represent from 25% to 45% by weight of said absorbent material.

According to these embodiments, therefore, said absorbent material may be composed of any one of about 15%, 16%, 17%, 18%, 19%, 20%, 21%, 22%, 23%, 24%, 25%, 26%, 27%, 28%, 29%, 350%, 31%, 32%, 33%, 34% or 35% by weight of said fumed silica could be in combination with any one of about 30%, 31%, 32%, 33%, 34%, 35%, 36%, 37%, 37%, 38%, 39%, 40%, 41%, 42%, 43%, 44%, 45%, 46%, 47%, 48%, 49%, or 50% by weight of said alginate, and in combination with any one of about 25%, 26%, 27%, 28%, 29%, 350%, 31%, 32%, 33%, 34%, 35%, 36%, 37%, 37%, 38%, 39%, 40%, 41%, 42%, 43%, 44%, or 45% by weight of said fumed silica. In an exemplary and non-limiting embodiment of the invention, said absorbent material may be composed of 20-30% by weight of said zeolites, 30-35% by weight of said fumed silica and 35-45% by weight of said alginate.

Liquid captation ability when the absorbent material is according to the above embodiment (said hydrated aluminosilicates represent from 15% to 35% by weight of said absorbent material, said alginate represents from 30% to 50% by weight of said absorbent material and said fumed silica represent from 25% to 45% by weight of said absorbent material) is between 90 and 140 ml of biological liquid or water for a weight of 10 g of said absorbent material.

In a preferred embodiment said hydrated aluminosilicates are zeolites, said fumed silica has a specific surface area from 100 to 500 m²/g or from 200 to 380 m²/g and said alginate is sodium alginate or calcium alginate.

In any case, therefore, the absorbent core of the article contains solely 100% biodegradable absorbent material, which can therefore be even completely composted or used for other purposes.

According to preferred embodiments of the invention, all components of the disposable absorbent article described herein are biodegradable and compostable. Therefore, according to one embodiment, the permeable upper layer of the disposable absorbent article of the invention is it also composed of compostable and/or biodegradable material, wherein this material comprises or consists in one or more from short cotton fibers and/or natural fibers, non-woven, polypropylene, biodegradable rayon, viscose.

According to a further embodiment, also said non-permeable lower layer is compostable and/or biodegradable and is composed of non-permeable biodegradable material made from plant products, like e.g. non-permeable compostable and/or biodegradable material made from sugar beet, vegetable cellulose, algae, fruit-processing waste, corn, like e.g. sodium polyacrylate and Superabsorbent Polymers (SAPs) commonly used in the state of the art.

Therefore, in a particularly advantageous embodiment, the disposable absorbent article of the invention, in which each layer is made of biodegradable and compostable material, is characterized in that it is fully compostable.

Besides what described above, the disposable absorbent article according to the invention can further comprise emollient and/or antibacterial and/or refreshing substances and/or fragrances commonly used in the art of absorbent articles.

The absorbent articles of the present invention will be suitable for any type of bodily fluid, such as, e.g., urine, blood, liquid feces or the like.

The absorbent article of the invention according to any one of the above-described embodiments can be made in the form of (adult) diaper, (infant/toddler) diaper, feminine hygiene absorbent article, absorbent towel.

As absorbent towel there are meant, e.g., underpads for beds, cots, seats and absorbent towels for bodily fluids.

According to another aspect, the invention relates to a process for the preparation of a disposable absorbent article wherein an absorbent core comprised of one or more silicon-based products selected from: hydrated aluminosilicates and fumed silica, optionally in combination with other silica-based compounds and/or natural silica and/or with alginate, is placed between a permeable upper layer and a non-permeable lower layer and wherein said absorbent core is prepared according to the following steps:

a. a volume of absorbent material consisting of said hydrated aluminosilicates and fumed silica, optionally in combination with other silica-based compounds and/or natural silica and/or in combination with alginate, is mixed and rehydrated with 2-4 volumes of water;

b. the mixture thus obtained is dried under vacuum at a pressure of between −0.8 and −1 bar and at a temperature comprised between 150° C. and 220° C. for a period of from 1.5 to 3 hours.

The mixing at step a. will be carried out in a suitable mixer. For the purposes of the invention, any suitable mixer known to a person skilled in the art could be used, like e.g. a concrete mixer-type mixer, in order to obtain a homogeneous compound.

The preparation of the absorbent core according to the process described herein entails a series of advantages, as fumed silica are normally highly volatile and therefore more difficult to insert into an absorbent article, whereas, following the above-described process, these deposit onto the other components of the absorbent material and is not volatile anymore, clearly making the preparation of the absorbent article simpler.

Moreover, the above-described treatment gives a greater absorption ability to the end mixture, compared to the starting materials mixed prior to the process (see Examples section).

According to a particular embodiment of the above-described invention, said hydrated aluminosilicates and fumed silica and optionally said alginate represent at least 80% by weight, 90% by weight, 95% by weight, 98% by weight of said absorbent material. In a particular embodiment of the invention the absorbent material may consist in a mixture of one or more hydrated aluminosilicates and one or more fumed silica without further materials added.

As indicated above, therefore, according to the invention one or more hydrated aluminosilicates and one or more fumed silica, in combination with other silica-based compounds and/or other minerals, can constitute the absorbent material for the preparation of the article according to the above-indicated process.

In particular, the other silica-based compounds can be selected in the group comprising

silicon dioxide SiO₂ bonded to other atoms Aluminum, Iron, Magnesium, Calcium, Sodium, Potassium.

Suitable to the ends of the carrying out of the present invention are aluminosilicates belonging to the family of ANALCIME, CHABASITE, GISMONDINE, HEULANDITE, NATROLITE, STILBITE, and zeolites.

Therefore, according to the invention the hydrated aluminosilicates can be selected, in any of the embodiments described herein, from natural or synthetic zeolites. Silica suitable for the carrying out of the invention are, e.g., fumed silica having a specific surface area from 100 to 500 m²/g, in particular from 200 and 380 m²/g (extremes included). A non-limiting example of suitable silica are the AEROSIL® silica, which are fumed silica available on the market, normally used also as pharmaceutical excipients, and therefore deemed suitable for human alimentary use. Therefore, for the carrying out of the invention silica within the above-indicated specific surface range, like, e.g., AEROSIL ® 150, 200, 250, 300, 380 or alike products, could be used. According to the invention, in any embodiment described herein there may be used, e.g., fumed silica having a specific surface area from 100 to 500 m²/g or from 200 to 380 m²/g.

According to what described above, in a particular embodiment, the hydrated aluminosilicates are selected from natural or synthetic zeolites and the fumed silica are selected from fumed silica having a specific surface area from 100 to 500 m²/g or from 200 to 380 m²/g.

In case the absorbent material be comprised of zeolites and fumed silica, such components could be present in said material in different mutual ratios.

For instance, according to various embodiments, absorbent materials are possible wherein said zeolites represent from 50% to 70% by weight of said absorbent material and said fumed silica represent from 30 al 50% by weight of said absorbent material or wherein said zeolites represent from 55% al 65% by weight of said absorbent material and said fumed silica represent from 35 al 45% by weight of said absorbent material, or wherein said zeolites represent about 60% by weight of said absorbent material and said fumed silica represent about 40% by weight of said absorbent material.

According to a further embodiment, as mentioned above, the absorbent core of the article contains also an alginate.

A non-limiting example of alginate according to the invention is represented by sodium alginate or calcium alginate.

Such alginate may be in the presence or in the absence of said other silica-based compounds and/or natural silica as described above.

Therefore, in a further embodiment, the article made according to the process as above-described will also comprise an alginate in a variable amount comprised between 30% and 50% by weight of said absorbent material.

Such alginate may be in the presence or in the absence of said other silica-based compounds and/or natural silica as described above.

According to these embodiments, therefore, when alginate is present, said hydrated aluminosilicates represent from 15% to 35% by weight of said absorbent material and said alginate represents from 30% to 50% by weight of said absorbent material and said fumed silica represent from 25% to 45% by weight of said absorbent material.

According to these embodiments, therefore, said absorbent material may be composed of any one of about 15%, 16%, 17%, 18%, 19%, 20%, 21%, 22%, 23%, 24%, 25%, 26%, 27%, 28%, 29%, 350%, 31%, 32%, 33%, 34% or 35% by weight of said fumed silica could be, in combination with any one of about 30%, 31%, 32%, 33%, 34%, 35%, 36%, 37%, 37%, 38%, 39%, 40%, 41%, 42%, 43%, 44%, 45%, 46%, 47%, 48%, 49%, or 50% by weight of said alginate, and in combination with any one of about 25%, 26%, 27%, 28%, 29%, 350%, 31%, 32%, 33%, 34%, 35%, 36%, 37%, 37%, 38%, 39%, 40%, 41%, 42%, 43%, 44% or 45% by weight of said fumed silica. In an exemplary and non-limiting embodiment of the invention, said absorbent material may be composed by 20-30% by weight of said zeolites, by 30-35% by weight of said fumed silica and by 35-45% by weight of said alginate. The liquid captation ability when the absorbent material is according to the above embodiment (said hydrated aluminosilicates represent from 15% to 35% by weight of said absorbent material, said alginate represents from 30% to 50% by weight of said absorbent material and said fumed silica represent from 25% to 45% by weight of said absorbent material) is between 90 and 140 ml of biological liquid or water for a weight of 10 g of said absorbent material.

In a preferred embodiment, said hydrated aluminosilicates are zeolites, said fumed silica has a specific surface area from 100 to 500 m²/g or from 200 to 380 m²/g and said alginate is sodium alginate or calcium alginate.

In any case therefore, the absorbent core of the article contains solely 100% biodegradable absorbent material, which therefore can also be completely composted or used for other purposes.

In a particular embodiment, in order to make a highly biodegradable and compostable article, the permeable upper layer is composed of compostable and/or biodegradable material comprising or consisting of one or more from short cotton fibers and/or natural fibers, non-woven, polypropylene, biodegradable rayon, viscose.

In a preferred embodiment, also said non-permeable lower layer is compostable and/or biodegradable and is composed of non-permeable biodegradable material made from plant products, like e.g. sugar beet, vegetable cellulose, algae, fruit-processing waste, corn.

Object of the invention is also a disposable absorbent article obtainable by the process according to any one of the above-described embodiments.

Moreover, the invention also relates to a process for the preparation of an absorbent mixture comprising the following steps:

a. a volume of absorbent material consisting of hydrated aluminosilicates and fumed silica, optionally in combination with other silica-based compounds and/or natural silica and/or in combination with alginate, is mixed and rehydrated with 2-4 volumes of water;

b. the mixture thus obtained is dried under vacuum at a pressure of between −0.8 and −1 bar and at a temperature comprised between 150° C. and 220° C. for a period of from 1.5 to 3 hours.

The mixing at step a. will be carried out in a suitable mixer. For the purposes of the invention any suitable mixer known to a technician in the field could be used, like e.g. a concrete mixer-type mixer, in order to obtain a homogeneous compound.

All of the materials described above for the preparation of the absorbent material and for the absorbent material of the disposable article of the present invention are suitable for the carrying out of the process for the preparation of the absorbent mixture described herein.

According to one embodiment of the invention, said hydrated aluminosilicates and fumed silica and optionally said alginate represent at least 80% by weight, 90% by weight, 95% by weight, 98% by weight of said absorbent material.

According to a particular embodiment, said absorbent mixture consists of a mixture of one or more hydrated aluminosilicates and one or more fumed silica.

In a particular embodiment, the hydrated aluminosilicates are selected from natural or synthetic zeolites.

According to a further embodiment, said fumed silica can be selected from fumed silica having a specific surface area from 100 to 500 m²/g or from 200 to 380 m²/g.

According to a particular embodiment, the mixture will consist of hydrated aluminosilicates selected from natural or synthetic zeolites and fumed silica selected from fumed silica having a specific surface area from 100 to 500 m²/g or from 200 to 380 m²/g.

For the carrying out of the invention, therefore, silica within the above-indicated range of specific surface, like, e.g., AEROSIL ® 150, 200, 250, 300, 380 or analogous products could be used. When the mixture consists of zeolites and fumed silica, various compositions thereof are available, like e.g. compositions wherein said zeolites represent from 50% to 70% by weight of said absorbent material and said fumed silica represent from 30 to 50% by weight of said absorbent material, or wherein said zeolites represent from 55% to 65% by weight of said absorbent material and said fumed silica represent from 35 to 45% by weight of said absorbent material, or wherein said zeolites represent about 60% by weight of said absorbent material and said fumed silica represent about 40% by weight of said absorbent material.

In a further embodiment, the mixture obtainable with the above-described process will also comprise an alginate in a variable amount of from 30% to 50% by weight of said absorbent material.

Such alginate, which according to a non-limiting example can be sodium alginate or calcium alginate, can be in the presence or in the absence of said other silica-based compounds and/or natural silica as described above.

According to these embodiments, therefore, when alginate is present, said hydrated aluminosilicates represent from 15% to 35% by weight of said absorbent mixture and said alginate represents from 30% to 50% by weight of said absorbent mixture and said fumed silica represent from 25% to 45% by weight of said absorbent mixture.

According to these embodiments, therefore, said absorbent mixture can be composed of any one of about 15%, 16%, 17%, 18%, 19%, 20%, 21%, 22%, 23%, 24%, 25%, 26%, 27%, 28%, 29%, 350%, 31%, 32%, 33%, 34% or 35% by weight of said fumed silica could be, in combination with any one of about 30%, 31%, 32%, 33%, 34%, 35%, 36%, 37%, 37%, 38%, 39%, 40%, 41%, 42%, 43%, 44%, 45%, 46%, 47%, 48%, 49%, or 50% by weight of said alginate, and in combination with any one of about 25%, 26%, 27%, 28%, 29%, 350%, 31%, 32%, 33%, 34%, 35%, 36%, 37%, 37%, 38%, 39%, 40%, 41%, 42%, 43%, 44%, or 45% by weight of said fumed silica. In an exemplary and non-limiting embodiment of the invention, said absorbent mixture can be composed of 20-30% by weight of said zeolites, 30-35% by weight of said fumed silica and 35-45% by weight of said alginate. The liquid captation ability when the mixture is prepared according to the above embodiment (said hydrated aluminosilicates represent from 15% to 35% by weight of said absorbent mixture, said alginate represents from 30% to 50% by weight of said absorbent mixture and said fumed silica represent from 25% to 45% by weight of said absorbent mixture) is from 90 and 140 ml of biological liquid or water for a weight of 10 g of said mixture.

In a preferred embodiment said hydrated aluminosilicates are zeolites, said fumed silica has a specific surface area from 100 to 500 m²/g or from 200 to 380 m²/g and said alginate is sodium alginate or calcium alginate.

Consequently, the invention also relates to an absorbent mixture obtainable by the process according to any one of the above-described embodiments.

The absorbent mixtures and the absorbent materials prepared according to the processes described herein are advantageously useful compared to the mere mixture of powders for the preparation of disposable absorbent articles, as fumed silica, which are extremely volatile, with the described process sediment on the other materials and is not volatile anymore. This feature of course makes the mixtures obtained with the above-described processes more suitable to the preparation of disposable absorbent articles.

Moreover, the absorption ability of the absorbent mixtures and materials prepared according to the processes of the invention increases sensibly compared to the absorption abilities of the powders mixed and not treated with the processes described herein.

The authors of the present invention performed some comparative assays, in which absorbent materials according to the invention and absorbent polymers commonly used in the preparation of absorbent articles such as diapers, like, e.g., absorbent gels available on the market under the name of Lysorb, were compared, and detected a greater absorption ability of the absorbent material (or absorbent mixture) and a greater ability to incorporate liquids, with an entailed improved sensation of dryness for the user by the absorbent material (or absorbent mixture) according to the invention.

### EXAMPLES

The absorbent mixture was prepared by mixing various ratios of fumed silica and hydrated aluminosilicates like those described in the present application, and the results were comparable. By way of example, a particular preparation of the absorbent mixture is reported hereinafter even though, as mentioned hereto, other relative ratios between fumed silica and hydrated aluminosilicates such as the ones specified above, i.e., wherein hydrated aluminosilicates represent from 50% to 70% by weight of the absorbent mixture and fumed silica represent from 30 to 50% by weight of the absorbent mixture; hydrated aluminosilicates represent from 55% to 65% by weight of the absorbent mixture and fumed silica represent from 35 to 45% by weight of the absorbent mixture; hydrated aluminosilicates represent about 60% by weight of the absorbent mixture and fumed silica represent about 40% by weight of the absorbent mixture.

### Example 1

Preparation of an Absorbent Mixture or of the Absorbent Material Contained in the Disposable Article According to the Invention

A mixture consisting of about 60% by weight of hydrated aluminosilicates as described in the present application and by about 40% of fumed silica, such as those described in the present application, was prepared.

The product was mixed and rehydrated with about 3 volumes of water per 1 volume of powders and the mixture was then thoroughly mixed in a concrete mixer-type mixer in order to obtain a homogeneous compound.

The mixture thus obtained was introduced into a drier under vacuum at the pressure of about −1 bar and at the temperature of 180° C.

The absorbent mixture thus obtained has two particular features; first of all the fumed silica, by depositing on the aluminosilicates, is not volatile anymore and therefore the mixture can easily be used for the preparation of disposable absorbent products such as those described in the present description; moreover, the absorption grade of the resulting mixture improves decidedly, of about +18.5%, compared to powders mixed prior to the treatment.

Finally, the product thus obtained is sterile.

### Example 2

Comparative Test Between the Absorption Ability of the Absorbent Mixture or of the Absorbent Material Contained in the Disposable Article According to the Invention and the Absorption Ability of Absorbent Polymers Available on the Market.

The test described herein was repeated 5 times.

Alike volumes of the mixture prepared according to test 1 and of the superabsorbent polymer available on the market under the name of Lysorb were compared.

Commercial polymer density=1

Density of absorbent mixture according to the invention=1.3

Absorption ability of 50 ml of commercial polymer=11 g H₂O

Absorption ability of 50 ml of absorbent mixture according to the invention=12.3 g H₂O

### Example 3

Comparative Absorption Test with Absorbent Paper Between the Absorbent Mixture or the Absorbent Material Contained in the Disposable Article According to the Invention and Absorbent Polymers Available on the Market.

The test described herein was repeated 5 times.

Water-saturated equal volumes of the mixture prepared according to test 1 and of the superabsorbent polymer available on the market under the name of Lysorb were compared.

The mixture of the invention, since the liquid remains entrapped in silica channels, has a practically nil liquid release when the product is saturated, whereas the commercial polymer, being a gel, behaves as a sponge and, by exerting a light pressure thereon, water release by the polymer occurs.

This feature is reflected by a greater sensation of dryness following use of the mixture of the invention, compared to the use of absorbent gels.

### Example 4

Preparation of a Further Absorbent Mixture or of the Further Absorbent Material Contained in the Disposable Article According to the Invention

A mixture was prepared, consisting of about 25% by weight of hydrated aluminosilicates as described in the present application, and about 35% of fumed silica as those described in the present application and about 40% of alginate as described in the present application.

The product was mixed and rehydrated with about 3 volumes of water per 1 volume of powders and the mixture was then thoroughly mixed in a concrete mixer-type mixer in order to obtain a homogeneous product.

The mixture thus obtained was introduced in a dryer under vacuum at the pressure of about −1 bar and at the temperature of about 180° C.

The absorbent mixture thus obtained has two peculiar features; first of all, the fumed silica, by depositing on aluminosilicates and on alginate, is not volatile anymore, and therefore the mixture can easily be used for the preparation of disposable absorbent products such as those described in the present description; moreover, the absorption grade of the resulting mixture further increases compared to that of the mixture prepared according to Example 1.

In this case as well, the product thus obtained is sterile.

The formulation as described herein enables to obtain a 12-14-fold absorption volume for biological fluids or water.

1. A disposable absorbent article comprising a permeable upper layer, a non-permeable lower layer and an absorbent core placed between the upper layer and the lower layer wherein said core contains, as sole absorbent material, one or more silicon-based products selected from the group consisting of: hydrated aluminosilicates and fumed silica, optionally in combination with other silica-based compounds and/or natural silica and/or in combination with alginate.

2. The disposable absorbent article according to claim 1 wherein said hydrated aluminosilicates and fumed silica, optionally in combination with said alginate, represent at least 80% by weight, 90% by weight, 95% by weight, or 98% by weight of said absorbent material.

3. The disposable absorbent article according to claim 1 wherein said absorbent material consists of a mixture of one or more hydrated aluminosilicates, and one or more fumed silica.

4. The disposable absorbent article according to claim 1 wherein said hydrated aluminosilicates are selected from group consisting of natural or synthetic zeolites.

5. The disposable absorbent article according to claim 1 wherein said fumed silica has a specific surface area from 100 to 500 m²/g or from 200 to 380 m²/g.

6. The disposable absorbent article according to any claim 1 wherein said alginate is from 30% to 50% by weight of said absorbent material.

7. The disposable absorbent article according to claim 1 wherein said alginate is selected from the group consisting of sodium alginate and calcium alginate.

8. The disposable absorbent article according to claim 1 wherein said hydrated aluminosilicates represent from 15% to 35% by weight of said absorbent material, said alginate represents from 30% to 50% by weight of said absorbent material, and said fumed silica represents from 25% to 45% by weight of said absorbent mixture.

9. The disposable absorbent article according to claim 8 wherein said hydrated aluminosilicates are zeolites, said fumed silica has a specific surface area from 100 to 500 m²/g or from 200 to 380 m²/g, and said alginate is sodium alginate or calcium alginate.

10. The disposable absorbent article according to claim 1 wherein said permeable upper layer is composed of compostable and/or biodegradable material comprising one or more from short cotton fibers and/or natural fibers, non-woven, polypropylene, biodegradable rayon, viscose.

11. The disposable absorbent article according to claim 1 wherein said non-permeable lower layer is compostable and/or biodegradable and is composed of non-permeable biodegradable material made from plant products.

12. The disposable absorbent article according to claim 11 wherein said non-permeable compostable and/or biodegradable material is made from sugar beet, vegetable cellulose, algae, fruit-processing waste, corn.

13. The disposable absorbent article according to claim 1, wherein it is fully compostable.

14. The disposable absorbent article according to claim 1 further comprising emollient and/or antibacterial and/or refreshing substances and/or fragrances.

15. The disposable absorbent article according to claim 1 wherein said article is in the form of an adult diaper, infant/toddler diaper, feminine hygiene absorbent article, or absorbent towel.

16. A process for the preparation of a disposable absorbent article wherein an absorbent core comprised of one or more silicon-based products selected from: hydrated aluminosilicates and fumed silica, optionally in combination with other silica-based compounds and/or natural silica and optionally with alginate, is placed between a permeable upper layer and a non-permeable lower layer and wherein said absorbent core is prepared according to the following steps:
a. a volume of absorbent material consisting of said hydrated aluminosilicates and fumed silica, optionally in combination with other silica-based compounds and/or natural silica and/or with alginate, is mixed and rehydrated with 2-4 volumes of water;
b. the mixture thus obtained is dried under vacuum at a pressure of between −0.8 and −1 bar and at a temperature comprised between 150° C. and 220° C. for a period of from 1.5 to 3 hours.

17. The process according to claim 16 wherein said hydrated aluminosilicates and fumed silica, and optionally said alginate,. represent at least 80% by weight, 90% by weight, 95% by weight, or 98% by weight of said absorbent material.

18. The process according to claim 16 wherein said absorbent material consists of a mixture of one or more hydrated aluminosilicates, and one or more fumed silica.

19. The process according to claim 16 wherein said hydrated aluminosilicates are selected from the group consisting of natural or synthetic zeolites.

20. The process according to claim 16 wherein said fumed silica has a specific surface area from 100 to 500 m²/g or from 200 to 380 m²/g.

21. The process according to claim 16 wherein said alginate is from 30% to 50% by weight of said absorbent material.

22. The process according to claim 16 wherein said alginate is selected from the group consisting of sodium alginate and calcium alginate.

23. The process according to claim 16 wherein said hydrated aluminosilicates represent from 15% to 35% by weight of said absorbent material, said alginate represents from 30% to 50% by weight of said absorbent material, and said fumed silica represents from 25% to 45% by weight of said absorbent mixture.

24. The process according to claim 23 wherein said hydrated aluminosilicates are zeolites, said fumed silica has a specific surface area from 100 to 500 m²/g or from 200 to 380 m²/g, and said alginate is sodium alginate or calcium alginate.

25. The process according to claim 17 wherein said permeable upper layer is composed of compostable and/or biodegradable material comprising one or more selected from the group consisting of short cotton fibers and/or natural fibers, non-woven, polypropylene, biodegradable rayon, and viscose.

26. The process according to claim 17 wherein said non-permeable lower layer is compostable and/or biodegradable, and is composed of non-permeable biodegradable material made from plant products.

27. The process according to claim 26 wherein said non-permeable compostable and/or biodegradable material is made from sugar beet, vegetable cellulose, algae, fruit-processing waste, corn.

28. A disposable absorbent article obtainable by the process according to claim 17.

29. A process for the preparation of an absorbent mixture comprising the following steps:
a. a volume of absorbent material consisting of hydrated aluminosilicates and fumed silica, optionally in combination with other silica-based compounds and/or natural silica and/or in combination with alginate, is mixed and rehydrated with 2-4 volumes;
b. the mixture thus obtained is dried under vacuum at a pressure of between −0.8 and −1 bar and at a temperature comprised between 150° C. and 220° C. for a period of from 1.5 to 3 hours.

30. The process according to claim 29 wherein said hydrated aluminosilicates and fumed silica, and optionally said alginate, represent at least 80% by weight, 90% by weight, 95% by weight, or 98% by weight of said absorbent material.

31. The process according to claim 29 wherein said absorbent material consists of a mixture of one or more hydrated aluminosilicates, and one or more fumed silica.

32. The process according to claim 29 wherein said hydrated aluminosilicates are selected from the group consisting of natural or synthetic zeolites.

33. The process according to claim 29 wherein said fumed silica has a specific surface area from 100 to 500 m²/g or from 200 to 380 m²/g.

34. The process according to claim 29 wherein said alginate is from 30% to 50% by weight of said absorbent material.

35. The process according to claim 29 wherein said alginate is selected from the group consisting of sodium alginate and calcium alginate.

36. The process according to claim 29 wherein said hydrated aluminosilicates represent from 15% to 35% by weight of said absorbent material, said alginate represents from 30% to 50% by weight of said absorbent material, and said fumed silica represents from 25% to 45% by weight of said absorbent mixture.

37. The process according to claim 36 wherein said hydrated aluminosilicates are zeolites, said fumed silica has a specific surface area from 100 to 500 m²/g or from 200 to 380 m²/g, and said alginate is sodium alginate or calcium alginate.

38. Absorbent mixture obtainable by the process according to claim 29.
